(19) United States
(12) Patent Application Publication
(10) Pub. No.: US 2007/0015814 A1
(43) Pub. Date: Jan. 18, 2007

(54) Parp Modulators and Treatment of Cancer

(76) Inventors: Ernest Kun, Mill Valley, CA (US); Jerome Mendeleyev, San Francisco, CA (US); Pal Bauer, Budapest (HU)

Correspondence Address: WILSON SONSINI GOODRICH & ROSATI, 650 PAGE MILL ROAD, PALO ALTO, CA, 94304-1050, US

(21) Appl. No.: 11/423,685

(22) Filed: Jun. 12, 2006

Related U.S. Application Data
(60) Provisional application No. 60/689,178, filed on Jun. 10, 2005.

(51) Int. Cl. A61K 31/404; A61K 31/366

(52) U.S. Cl. 514/415; 514/419; 514/457

(57) ABSTRACT

The invention relates to a method of modulating poly(ADP-ribose)polymerase-1 (PARP-1) activity in a mammal comprising administering to a mammal an effective amount of an organic aromatic compound having from 4 to about 35 carbon atoms, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1 and does not contain benzamide or lactam substituents. In particular, substituted benzopyrones and substituted indoles and their pharmaceutical compositions containing such compounds that modulate the activity of PARP-1, are described. The invention is also directed to the composition of matter, kits and methods for their therapeutic and/or prophylactic use in treating diseases and disorders described herein, by administering effective amounts of such compounds. Preferably, the compositions and methods provided herein inhibit PARP activity.

### CROSS-REFERENCE

This application claims priority to U.S. Provisional Application No. 60/689,178, filed Jun. 10, 2005, which is incorporated herein by reference in its entirety.

### STATEMENT AS TO FEDERALLY SPONSORED RESEARCH

This invention was in part made with the support of the United States government under NIH grants HL 59693 and HL 35561.

### BACKGROUND OF THE INVENTION

PARP (poly-ADP ribose polymerase) participates in a variety of DNA-related functions including gene amplification, cell division, differentiation, apoptosis, DNA base excision repair and also has effects on telomere length and chromosome stability (d'Adda di Fagagna et al, 1999, Nature Gen., 23(1): 76-80). Oxidative stress-induced overactivation of PARP consumes NAD+ and consequently ATP, culminating in cell dysfunction or necrosis. This cellular suicide mechanism has been implicated in the pathomechanism of stroke, myocardial ischemia, diabetes, diabetes-associated cardiovascular dysfunction, shock, traumatic central nervous system injury, arthritis, colitis, allergic encephalomyelitis, and various forms of inflammation. PARP has also been shown to associate with and regulate the function of several transcription factors. The multiple functions of PARP make it a target for a variety of serious conditions including various types of cancer and neurodegenerative diseases.

PARP-inhibition therapy represents an effective approach to treat a variety of diseases. In cancer patients, PARP inhibition may increase the therapeutic benefits of radiation and chemotherapy. Targeting PARP may prevent tumor cells from repairing DNA themselves and developing drug resistance, which may make them more sensitive to cancer therapies. PARP inhibitors have demonstrated the ability to increase the effect of various chemotherapeutic agents (e.g. methylating agents, DNA topoisomerase inhibitors, cisplatin etc.), as well as radiation, against a broad spectrum of tumors (e.g. glioma, melanoma, lymphoma, colorectal cancer, head and neck tumors).

The incidence of breast cancer in women rose from 100.5 cases per 100,000 population in 1991 to 117.2 cases per 100,000 population in 2001; an average increase of 1.4% per annum. Women carrying faults in the BRCA1 and 2 genes have up to an 85% chance of developing breast cancer by the age of 70. PARP inhibitors may be effective in killing tumor cells in people who have faults in BRCA1 and BRCA2. PARP inhibitors have the potential to help the specific subset of patients who have mutations in these genes. These mutations predispose patients to early-onset of cancer and have been found in breast, ovarian, prostate and pancreatic cancers.

PARP inhibitors can be combined with other chemotherapeutics such as, irinotecan or temozolomide to improve the treatment of a number of cancers such as colorectal and gastric cancers, and melanoma and glioma, respectively. PARP inhibitors can be combined with irinotecan to treat advanced colorectal cancer. Approximately 146,000 new cases of colorectal cancer are expected in the US in 2004 and of this 60-70% are expected to be in advanced stages.

PARP inhibitors have been designed as analogs of benzamides, which bind competitively with the natural substrate NAD in the catalytic site of PARP. This includes a variety of cyclic benzamide analogs (i.e., lactams) which are potent inhibitors at the NAD site. However, the approach of using benzamide analogs has been limited in effect in vivo. These benzamides and lactams can bind to other NAD-utilizing enzymes, which are ubiquitous, and generate side effects and affect cell viability, metabolism and DNA synthesis. As an example see Milan et al, (1984) “Inhibitors of Poly (Adenosine Diphosphate Ribose) Synthesis: Effect on Other Metabolic Processes”, Science 223: 589-91. Thus, there remains a need for compounds that inhibit PARP activity which produce potent and reliable effects with fewer side effects with respect to inhibiting PARP activity and treating the related diseases and conditions.

Accordingly, the present invention provides compositions and methods for modulating PARP activity in a mammal suffering from a PARP mediated disease.

### SUMMARY OF THE INVENTION

The present invention relates to a pharmaceutical composition comprising: (i) an effective amount of an organic aromatic compound having from 4 to about 35 carbon atoms that modulates PARP-1 activity in a mammal, wherein said organic aromatic compound is (a) capable of binding the arginine-34 moiety located in Zinc finger-1 of the PARP-1 enzyme and (b) wherein said organic aromatic compound has electron donating capabilities such that it's π-electron system will interact with the positively charged (cationic) guanidinium moiety of the specific arginine-34 residue of the Zinc-1 finger of PARP-1, (c) wherein said aromatic compound contains a heterocyclic ring containing a nitrogen atom, (d) said ring does not contain a carbonyl moiety; and (ii) a pharmaceutically acceptable carrier, excipient and/or diluents. Preferably, the compositions of the present invention inhibit PARP activity.

The invention also relates to a method of modulating PARP-1 activity in a mammal comprising administering to a mammal an effective amount of an organic aromatic compound having from 4 to about 35 carbon atoms, wherein said organic aromatic compound is capable of binding the arginine-34 moiety located in Zinc finger-1 of the PARP-1 enzyme and wherein said organic aromatic compound has electron donating capabilities such that its π-electron system will interact with the positively charged (cationic) guanidinium moiety of the specific arginine-34 residue of the Zinc-1 finger of PARP-1 wherein said aromatic compound contains a heterocyclic ring containing a nitrogen atom, said ring does not contain a carbonyl moiety and does not contain a lactam structure and is not a benzamide analog and not an analog of NAD. The compounds of the present invention act via the ATP binding site and may or may not interact with the NAD site. Preferably, the methods of the present invention inhibit PARP activity.

The invention specifically relates to a method of modulating PARP-1 activity in a mammal comprising administering to a mammal an effective amount of an organic aromatic compound of formula I, 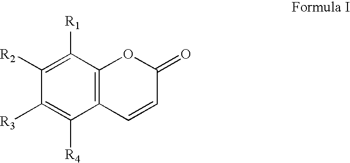
wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1; wherein R₁, R₂, R₃ and R₄ are independently selected from the group consisting of H, halogen, optionally substituted hydroxy, substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof. Preferably, this methods of the present invention inhibit PARP activity.

In a preferred embodiment, the invention relates to compound of formula I 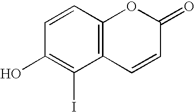
wherein R₁, R₂ is H or a salt, solvate, isomer, tautomers, metabolite, or prodrug thereof.

Another aspect of the invention relates to a method of modulating PARP-1 activity in a mammal comprising administering to a mammal an effective amount of an organic aromatic compound of formula II, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc finger-1 of PARP-1 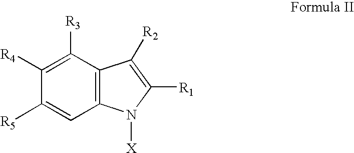
wherein R₁, R₂, R₃, R₄ and R₅ are independently selected from the group consisting of H, halogen, nitro, nitroso, optionally substituted hydroxy, optionally substituted lower alkyl, optionally substituted amine, optionally substituted phenyl, optionally substituted C₄-C₁₀ heteroaryl and optionally substituted C₃-C₈ cycloalkyl; X is H, N-oxide or optionally substituted alkyl or a salt, solvate, isomer, tautomers, metabolite, or prodrug thereof. Preferably, this methods of the present invention inhibit PARP activity.

In a preferred embodiment, the invention relates to a subset of compounds of formula II as shown in Formula IIa 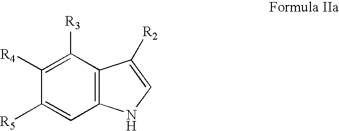
wherein R₁ and X is H and R₂, R₃, R₄ and R₅ are independently selected from the group consisting of halo, preferably, iodo, hydroxyl, nitro, nitroso, and optionally substituted amine such as aminoalkyl or a salt, solvate, isomer, tautomers, metabolite, or prodrug thereof.

A particularly preferred group of compounds of formula IIa is wherein R₂ is alkylamine, preferably propylamine.

Another preferred class of compounds of formula IIa is wherein R₃, R₄ or R₅ is halogen, preferably iodine.

Another preferred class of compounds of formula IIa is wherein R₃, R₄ or R₅ is hydroxyl.

One aspect of the invention is a method of treatment of a PARP mediated disease comprising administering to a subject in need thereof a therapeutically effective amount of an organic aromatic compound having from 4 to about 35 carbon atoms, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1 where when said aromatic compound contains a heterocyclic ring containing a nitrogen atom, said ring does not contain a carbonyl moiety.

Another aspect of the invention is a method of treatment of a PARP mediated disease comprising administering to a subject in need thereof a therapeutically effective amount of an organic aromatic compound of formula I, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1 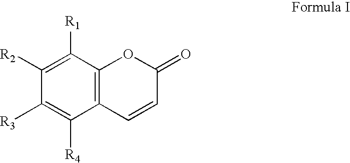
wherein R₁, R₂, R₃ and R₄ are independently selected from the group consisting of H, halogen, optionally substituted hydroxy, substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof.

Another aspect of the invention is a method of treatment of a PARP mediated disease comprising administering to a subject in need thereof a therapeutically effective amount of an organic aromatic compound of formula II, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1 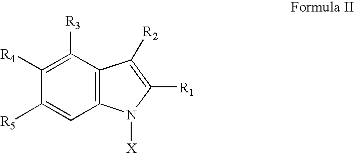
wherein R₁, R₂, R₃, R₄ and R₅ are independently selected from the group consisting of H, halogen, nitro, nitroso, optionally substituted hydroxy, optionally substituted lower alkyl, optionally substituted amine, optionally substituted phenyl, optionally substituted C₄-C₁₀ heteroaryl and optionally substituted C₃-C₈ cycloalkyl; X is H, N-oxide or optionally substituted alkyl or a salt, solvate, isomer, tautomers, metabolite, or prodrug thereof.

Particularly preferred examples of compounds of the present invention include, but are not limited to, the following:

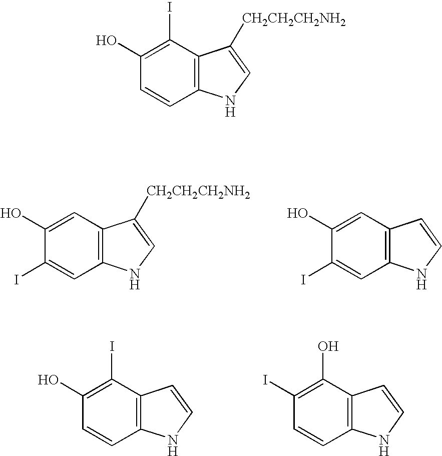

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph illustrating the enzymatic activities of wild type arginine-34 and arginine-138 mutant PARP-1.

FIG. 2 is a graph illustrating the effect of ATP on the PARP-1 activity of Jurkat cell nuclei.

FIG. 3 is a graph illustrating the effect of BCNU on the ATP sensitivity of PARP-1 activity of Jurkat cell nuclei.

FIG. 4 is a graph illustrating the effect of ATP on the glycohydrolase activity of Jurkat cell nuclear extract.

FIG. 5 is a graph illustrating the effect of the chain-length of the PAR polymer on the ATP sensitivity of purified PARG.

FIG. 6 is a graph illustrating the effect of ATP on PARG activity as a function of substrate (PAR) concentration.

FIG. 7 is a drawing that depicts one embodiment of an interaction between the aromatic π-system and the cationic guanidinium moeity of PARP-1 wherein X═OH or NH₂.

### DETAILED DESCRIPTION OF THE INVENTION

The term “alkyl” as used herein refers to straight- and branched-chain alkyl groups having one to eight carbon atoms. Exemplary alkyl groups include methyl (Me), ethyl, n-propyl, isopropyl, butyl, isobutyl, sec-butyl, tert-butyl (tBu), pentyl, isopentyl, tert-pentyl, hexyl, isohexyl, and the like. Substituted alkyls include aminoalkyl, hydroxyalkyl, alkoxyalkyl and the like. Substituted alkyls are also represented by an alkyl substituted with, e.g., a substituted or unsubstituted C₃-C₈ cycloalkyl, C₃-C₈ heterocycloalkyl, phenyl, or C₄-C₁₀ heteroaryl.

The term “aminoalkyl” refers to —CH₂—R—NH₂ where R is an alkyl group as defined above.

The term “cycloalkyl” refers to saturated carbocycles having from three to eight carbon atoms, including bicyclic and tricyclic cycloalkyl structures. Exemplary cycloalkyls include cyclopropyl, cyclobutyl, cyclopentyl, cyclohexyl, cycloheptyl, and the like.

The term “halogen” refers to chlorine, fluorine, bromine or iodine. The term “halo” represents chloro, fluoro, bromo or iodo. Most preferred embodiments of the present invention include iodo as the halo group.

The term “heteroaryl” refers to mono heterocyclic and poly heterocyclic unsaturated or aromatic ring structures. Examples of heterocyclic ring structures include furyl, thienyl, pyrrolyl, pyridyl, pyridinyl, pyrazolyl, imidazolyl, pyrazinyl, pyridazinyl, 1,2,3-triazinyl, 1,2,4-oxadiazolyl, 1,3,4-oxadiazolyl, 1-H-tetrazol-5-yl, indolyl, quinolinyl, benzofuranyl, benzothiophenyl (thianaphthenyl), and the like. Such moieties may be optionally substituted by one or more suitable substituents, for example, a substituent selected from a halogen (F, Cl, Br or I); lower alkyl; OH; NO₂; CN; CO₂H; O-lower alkyl; phenyl; phenyl-lower alkyl; CO₂ CH₃; CONH₂; OCH₂ CONH₂; NH₂; SO₂ NH₂ OCHF₂; CF₃; OCF₃; and the like. Such moieties may also be optionally substituted by a fused-ring structure or bridge, for example OCH₂—O.

The term “inhibits” and its grammatical conjugations, such as “inhibitory,” are not intended to require complete reduction in PARP activity. Such reduction is preferably by at least about 50%, at least about 75%, at least about 90%, and more preferably by at least about 95% of the activity of the molecule in the absence of the inhibitory effect, e.g., in the absence of an inhibitor, such as compounds I, II and/or their preferred embodiments of the invention. Most preferably, the term refers to an observable or measurable reduction in activity. In treatment scenarios, preferably the inhibition is sufficient to produce a therapeutic and/or prophylactic benefit in the condition being treated. The phrase “does not inhibit” or its grammatical conjugations do not require a complete lack of effect on the activity. For example, it refers to situations where there is less than about 20%, less than about 10%, and preferably less than about 5% of reduction in PARP activity in the presence of an inhibitor such as compounds I, II and/or their preferred embodiments of the invention.

The term “pharmaceutically acceptable salt” refers to those salts which retain the biological effectiveness and properties of the compounds used in the present invention, and which are not biologically or otherwise undesirable. For example, a pharmaceutically acceptable salt does not interfere with the beneficial effect of the compound of the invention in treating a cancer.

The term “a pharmaceutically acceptable prodrug” refers to a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity).

The term “a pharmaceutically active metabolite” refers to a pharmacologically active product produced through metabolism in the body of a specified compound or salt thereof. After entry into the body, most drugs are substrates for chemical reactions that may change their physical properties and biologic effects. However, in some cases, metabolism of a drug is required for therapeutic effect.

The term “therapeutically effective amount” refers to an amount effective to achieve therapeutic or prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. The actual amount effective for a particular application will depend on the patient (e.g., age, weight, etc.), the condition being treated, and the route of administration. Determination of an effective amount is well within the capabilities of those skilled in the art. The effective amount for use in humans can be determined from animal models. For example, a dose for human can be formulated to achieve circulating and/or gastrointestinal concentrations that have been found to be effective in animals.

Compositions and Methods for PARP Inhibitors

The present invention relates to a method for inhibiting PARP-1 by binding organic molecules to arginine-34 which is located in Zn²⁺ finger 1 of the PARP-1 enzyme. It is known that arginine residues in protein can participate in ATP sensing (Ogura et al. (2004) J. Struct. Biol. 146:106-112) and arginine residues were identified in both Zn²⁺ fingers of PARP-1 without assignation of specific catalytic function (Molinet et al. (1993) EMBO J. 12:2109-2117; Ikeyama et al. (1990) J. Biol. Chem. 265:21907-21913). The point mutational analysis of arginine-34 shows that arginine substitution in arginine-34 by another amino acid such as glycine in Zn²⁺ finger 1 of PARP-1 does not affect the total enzymatic activity of PARP-1 but the inhibitory action of ATP is abolished. The mutation of arginine-138 by substitution with isoleucine in Zn²⁺ finger 2 of PARP-1 has negligible effect on the inhibitory action of ATP, confirming the observation that arginine-34 of Zn²⁺ finger 1 is the site of ATP interaction with PARP-1.

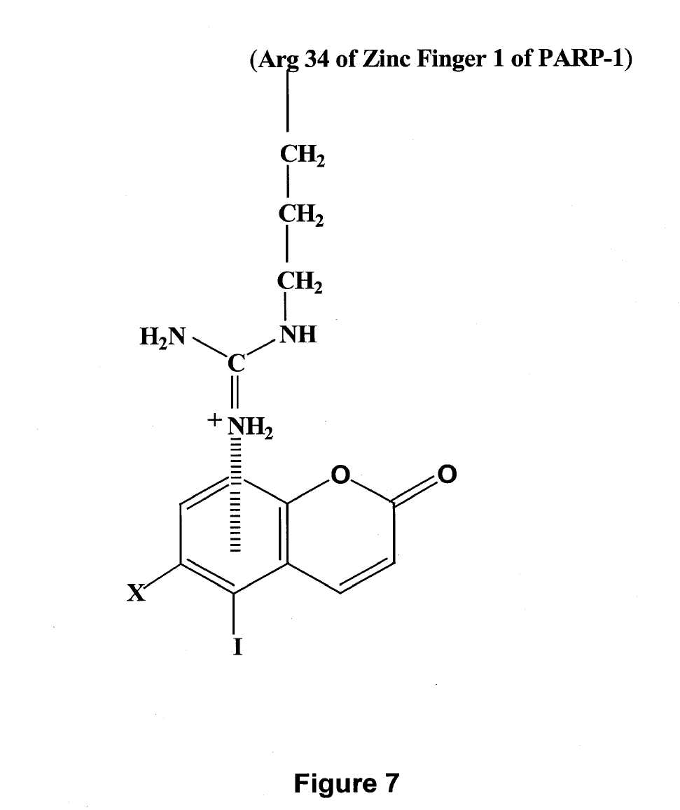

It is known that the guanidine moiety of arginine assumes central importance as a cation in cationic-π interactions (Zacharias et al. (2002) Trends in pharmacological Sciences 23:281-287; Woods et al. (2004) J. Proteome Res. 3:478-484). An aspect of this invention involves the inhibition of PARP-1 by the cationic-π interactions between the guanidine moiety of arginine and the π-system of the candidate molecules as depicted in FIG. 7 using either 5-iodo-6-hydroxybenzopyrone or 5-iodo-6-aminobenzopyrone as examples. The substitution of the aromatic ring with the electron donating substituents increases the electron density in the ring with subsequent increase in the cationic-π interaction with the guanidine moiety of arginine, thereby, increasing the inhibition of PARP-1 by use of such organic aromatic molecules. Inhibiting the activity of a PARP molecule includes reducing the activity of these molecules.

The inhibitory site at arginine-34 in the Zinc finger 1 of PARP-1 obviates the need to inhibit PARP-1 at the NAD catalytic site, thus removing the need to employ benzamides or analogous lactams which compete with NAD and thereby have drawbacks in vivo. The new aromatic electron-donating inhibitors at the arginine-34 site are a new class, a feature of which is that they designedly do not contain benzamide or lactam groups. The compounds of the invention are substituted 1,2-benzopyrones, indoles, or benzimidazoles, which do not contain fusion with a third ring (i.e., are not tricyclic) and do not contain a lactam group; and are not benzamide analogs, i.e., have no benzamide core.

The aromatic molecules that can serve as iπ-electron donors interacting with the arginine-34 cation can be divided into two categories: (1) interacting inhibitors (preferably candidates for anti-cancer drugs), and (2) physiologically occurring molecules bearing aromatic groups that temporally regulate PARP following metabolic demands of the cell. Selection of aromatic compounds can be determined by the reactivity with the arginine-34 site, and modification of aromatic systems can be determined by that reactivity. Typically, kinetic evidence for reactivity with arginine-34 consists of additive inhibition to that of ATP (T. C. Chou and P. Talalay, Adv. Enzyme Regul. 22:27 (1984)).

In some preferred embodiments of the present invention, aromatic π-system interacting with the arginine-34 cation includes 1,2-benzopyrone (coumarin) such as formula I, indole (Formula II) optionally substituted with iodine, or benzimidazole (Formula III) optionally substituted with iodine.

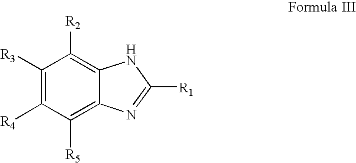

As indicated, the various moieties or functional groups for variables in the formulae may be optionally substituted by one or more suitable substituents. Exemplary substituents include a halogen (F, Cl, Br, or I), lower alkyl, —OH, —NO₂, —CN, —CO₂H, —O-lower alkyl, -phenyl, -phenyl-lower alkyl, —CO₂ CH₃, —CONH₂, —OCH₂ CONH₂, —NH₂, —SO₂ NH₂, haloalkyl (e.g., —CF₃, —CH₂ CF₃), —O-haloalkyl (e.g., —OCF₃, —OCHF₂), and the like. Preferably the halogen is an iodo group.

The invention relates to a method of modulating, preferably inhibiting PARP-1 activity in a mammal using an organic aromatic compound having from 4 to about 35 carbon atoms, including formula I, its preferred embodiment, formula II and/or its preferred embodiments, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc finger-1 of PARP-1 where when said aromatic compound contains a heterocyclic ring containing a nitrogen atom, said ring does not contain a carbonyl moiety. The invention is also directed to the therapeutic or prophylactic use of such compounds and methods of treating diseases and disorders that involve PARP activation.

Another aspect of the invention is a method of treatment of a PARP mediated disease comprising administering to a subject in need thereof a therapeutically effective amount of an organic aromatic compound of formula I, wherein said organic aromatic compound is capable of binding the arginine-34 moiety located in Zinc finger-1 of the PARP-1 enzyme and wherein said organic aromatic compound has electron donating capabilities such that it's iπ-electron system will interact with the positively charged (cationic) guanidinium moiety of the specific Arginine-34 residue of the Zinc finger-1 of PARP-1 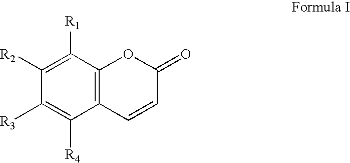
wherein R₁, R₂, R₃ and R₄ are independently selected from the group consisting of H, halogen, optionally substituted hydroxy, substituted amine, optionally substituted nitro, optionally substituted lower alkyl, optionally substituted phenyl, optionally substituted C₄-C₁₀ heteroaryl and optionally substituted C₃-C₈cycloalkyl or a salt, solvate, isomer, tautomers, metabolite, or prodrug thereof; and does not contain a lactam group, nor carries a lactam or benzamide substituent.

A preferred embodiment of formula I 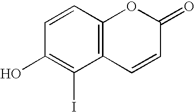
wherein R₁, R₂ is H or a salt, solvate, isomer, tautomers, metabolite, or prodrug thereof.

Another aspect of the invention is a method of treatment of a PARP mediated disease comprising administering to a subject in need thereof a therapeutically effective amount of an organic aromatic compound of formula II, wherein said organic aromatic compound is capable of binding the arginine-34 moiety located in Zinc finger-1 of the PARP-1 enzyme and wherein said organic aromatic compound has electron donating capabilities such that it's iπ-electron system will interact with the positively charged (cationic) guanidinium moiety of the specific arginine-34 residue of the Zinc finger-1 of PARP-1 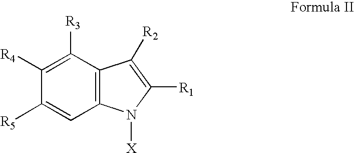
wherein R₁, R₂, R₃, R₄ and R₅ are independently selected from the group consisting of H, halogen, nitro, nitroso, optionally substituted hydroxy, optionally substituted lower alkyl, optionally substituted amine, optionally substituted nitro, optionally substituted phenyl, optionally substituted C₄-C₁₀ heteroaryl and optionally substituted C₃-C₈ cycloalkyl; X is H, N-oxide or optionally substituted alkyl or a salt, solvate, isomer, tautomers, metabolite, or prodrug thereof.

A preferred embodiment, a subset of compounds of formula II is shown in Formula IIa 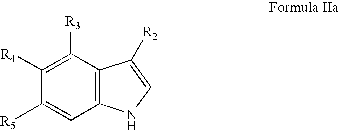
wherein R₁ and X is H and R₂, R₃, R₄ and R₅ are independently selected from the group consisting of iodo, hydroxyl, nitro, nitroso, and optionally substituted amine such as, aminoalkyl or a salt, solvate, isomer, tautomers, metabolite, or prodrug thereof.

A particularly preferred group of compounds of formula IIa is wherein R₂ is alkylamine, preferably propylamine.

Another preferred class of compounds of formula IIa is wherein R₃, R₄ or R₅ is halogen, preferably iodine.

Another preferred class of compounds of formula Ia is wherein R₃, R₄ or R₅ is hydroxyl.

Particularly preferred examples of compounds of the present invention include, but are not limited to, the following:

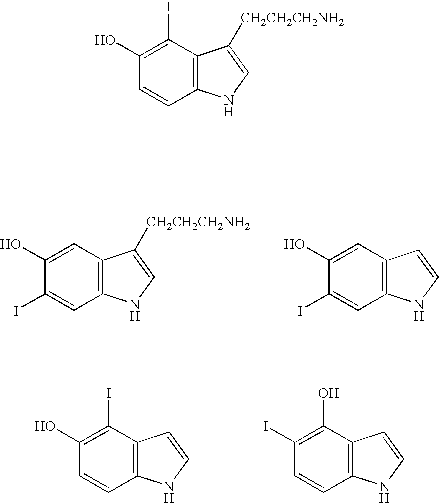

The compounds of the invention may exhibit the phenomenon of tautomerism. While Formula I, II and IIa cannot expressly depict all possible tautomeric forms, it is to be understood that Formula I, II and IIa are intended to represent any tautomeric form of the depicted compound and are not to be limited merely to a specific compound form depicted by the formula drawings. Some of the compounds of the invention may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

Additionally, the formulas are intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine. In addition to compounds of Formula I, II and Ia, the invention includes pharmaceutically acceptable prodrugs, pharmaceutically active metabolites, and pharmaceutically acceptable salts of such compounds and metabolites.

The compounds described herein can be synthesized using techniques known in the art. A variety of substituents can be introduced into the aromatic nuclei of 1,2-benzopyrone, indole, and benzimidazole. Typically, amino substituents can be introduced by way of standard nitration techniques, followed by reduction of such nitro groups to amino groups. Amino groups on the aromatic nuclei can be diazotized and converted to a variety of other groups such as halogens and hydroxyls by Sandmeyer-type processes. Additionally, halogenations can be performed directly on hydroxyl- and amino-substituted aromatic rings, giving di-substituted examples. Further, the halogen groups can be utilized as leaving groups to be replaced by reagents, such as organometallics, to introduce alkyl groups.

Poly (ADP-Ribose) Polymerase (PARP)

The poly (ADP-ribose) polymerase (PARP) is also known as poly (ADP-ribose) synthase and poly ADP-ribosyltransferase. PARP is an enzyme located in the nuclei of cells of various organs, including muscle, heart and brain cells. PARP catalyzes the formation of poly (ADP-ribose) polymers which can attach to nuclear proteins (as well as to itself) and thereby modify the activities of those proteins. The enzyme plays a role in enhancing DNA repair, and also possibly plays a role in regulating chromatin in the nuclei (for review see: D. D'amours et al. “Poly (ADP-ribosylation reactions in the regulation of nuclear functions,” Biochem. J. 342: 249-268 (1999)).

PARP-1 comprises an N-terminal DNA binding domain (DBD), an automodification domain and a C-terminal catalytic domain and various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interact with PARP-1 in this domain. The automodification domain contains a BRCT motif, one of the protein-protein interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), Yin Yang (YY) 1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly (ADP-ribose) metabolism could be related to a variety of cell regulatory functions.

The most studied member of this gene family is PARP1. The PARP1 gene product is expressed at high levels in the nuclei of cells and is dependent upon DNA damage for activation. Without being bound by any theory, it is believed that PARP1 binds to DNA single or double stranded breaks through an amino terminal DNA binding domain. The binding activates the carboxy terminal catalytic domain and results in the formation of polymers of ADP-ribose on target molecules. PARP1 is itself a target of poly ADP-ribosylation by virtue of a centrally located automodification domain. The ribosylation of PARP1 causes dissociation of the PARP1 molecules from the DNA. The entire process of binding, ribosylation, and dissociation occurs very rapidly. It has been suggested that this transient binding of PARP1 to sites of DNA damage results in the recruitment of DNA repair machinery or may act to suppress the recombination long enough for the recruitment of repair machinery.

Bauer et al. (Int. J. Oncol. 8, 239, 1996) demonstrated that poly ADP-ribosylation in cancer cells inhibits Ca²⁺—Mg²⁺ dependent DNAase, thereby allowing uncontrolled cancer replication. De-inhibition of the DNAase (by PARP-1 inhibition) may initiate DNA breakdown that is specific for cancer cells and induce apoptosis in cancer cells only. The physiologically existing dsDNAs are far superior coenzymes of PARP-1 to damaged DNAs, thus placing PARP-1 as a physiologically operative chromatin regulator in intact cells (Kun et al., J. Biol. Chem. 277, 39066, 2002), which functions differently in the cancerous phenotype.

The N-terminal DBD in PARP-1, extends from the initiator methionine to threonine-373 in human PARP. This domain has a molecular mass of approximately 42 kDa and contains two zinc fingers and two helix-turn-helix motifs. The DBD of PARP also contains a high proportion of basic residues, which may be involved in the interaction of the enzyme with DNA. PARP is a metalloenzyme that binds zinc molecules specifically. The zinc-binding sites are associated with a 29 kDa fragment of PARP derived from the limited proteolysis of the protein with trypsin. The association of PARP with zinc suggests that the enzyme possesses zinc fingers, which was later confirmed by sequence analysis of the cloned cDNA. Zinc finger 1 (F1) starts at cysteine-21 and ends at cysteine-56, while zinc finger 2 (F2) is found between cysteine-125 and cysteine-162. See D'amours et al., Op. Cit. (1999).

Not intending to be limited by one mechanism of action, one aspect of the inventions involves a bimodal action of ATP on the polyADP-ribose cycle i.e. its degradation site and specifically at the polyADP-ribose synthesis site. It deals with the action of ATP on isolated cell nuclei, which involves the inhibition of polyADP-ribosylation and also the action of ATP on a specific glycohydrolase that regulates the degradation of protein bound polyADP-ribose chains. Isolated cell nuclei also respond to both inhibition of poly(ADP-ribose) polymerase by ATP and activation of poly(ADP-ribose) glycohydolase by ATP, demonstrating that enzymological results can be extrapolated to cellular systems.

It has been demonstrated that there is an inhibition of PARP-1 by physiologic concentrations of ATP (or its non-hydrolysable analog) on Zn²⁺ finger 1 of PARP-1 (Kun et al., Biochemistry 43, 210, 2004); which is not in the NAD catalytic site. This inhibitory site was further identified by amino acid mutation of arginine residues in both Zn²⁺ fingers in order to pinpoint the ATP site (Bauer et al., Int. J. Mol. Med. 2005, accepted, now in press), which was found to be arginine-34 in Zn²⁺ finger 1. Since arginine residues (Zacharias et al., Trends in Pharmacol. Sci. 23, 281, 2002) can react with “aggressive” phosphates (e.g. ATP) and aromatics as well (e-donors) (Woods, J. Proteomic Res. 3, 478, 2004), the ATP “site” may co identify sites of aromatics that inhibit PARP-1 via reactivity through arginine-34 in Zn²⁺ finger 1. Thus, the PARP inhibitors of the present invention may be identifiable by their interaction with arginine-34 and kinetically identifiable by additivity to ATP inhibition. The arginine-34 selective PARP-1 inhibitors of the present invention can act directly on tumor cells due to the high PARP-1 activity of cancers which is a characteristic biochemical phenotype of cancers.

PARP Mediated Diseases

One aspect of the invention is a method of treatment of a PARP mediated disease comprising administering to a subject in need thereof a therapeutically effective amount of an organic aromatic compound having from 4 to about 35 carbon atoms, including formula I, its preferred embodiment, formula II and/or its preferred embodiments as mentioned above, wherein said organic aromatic compound is capable of binding the arginine-34 moiety located in Zinc finger-1 of the PARP-1 enzyme and wherein said organic aromatic compound has electron donating capabilities such that it's-π-electron system will interact with the positively charged (cationic) guanidinium moiety of the specific arginine-34 residue of the Zinc finger-1 of PARP-1 where when said aromatic compound contains a heterocyclic ring containing a nitrogen atom, said ring does not contain a carbonyl moiety.

Various PARP mediated diseases are, but not limited to, cancer types including adrenal cortical cancer, anal cancer, aplastic anemia, bile duct cancer, bladder cancer, bone cancer, bone metastasis, adult CNS brain tumors, children CNS brain tumors, breast cancer, Castleman disease, cervical cancer, childhood Non-Hodgkin's lymphoma, colon and rectum cancer, endometrial cancer, esophagus cancer, Ewing's family of tumors, eye cancer, gallbladder cancer, gastrointestianl carcinoid tumors, gastrointestinal stromal tumors, gestational trophoblastic disease, Hodgkin's disease, Kaposi'sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sacrcoma, vaginal cancer, vulvar cancer, and Waldenstrom's macroglobulinemia.

PARP mediated diseases include angiogenesis in cancers, inflammation, degenerative diseases, CNS diseases, autoimmune diseases, and viral diseases, including HIV. The compounds described herein are also useful in the modulation of cellular response to pathogens. The invention also provides methods to treat other PARP mediated diseases, such as, viral diseases. Some of the viral diseases are, but not limited to, human immunodeficiency virus (HIV), herpes simplex virus type-1 and 2 and cytomegalovirus (CMV), a dangerous co-infection of HIV.

Other PARP mediated diseases are, but not limited to, inflammatory bowel disorder, arthritis, hyperglycemia, diabetes, endotoxic shock or septic shock, peripheral nerve injuries, skin aging, epilepsy, stroke, Parkinson's disease, amyotrophic lateral sclerosis, Huntington's disease, schizophrenia, chronic pain, ischemia, neuronal loss following hypoxia, Alzheimer's disease, atherosclerosis, osteoarthritis, osteoporosis, muscular dystrophy, degenerative diseases of skeletal muscle involving replicative senescence, age-related macular degeneration, immune senescence, and other immune senescence diseases. In some embodiments, the compounds and methods described herein are used for modulation, preferably inhibition, of angiogenesis or inflammation.

Some examples of the PARP mediated diseases are set forth here, but without limiting the scope of the present invention, there may be other PARP mediated diseases known in the art and are within the scope of the present invention.

Examples of Cancer

Examples of cancers include, but are not limited to, lymphomas, carcinomas and hormone-dependent tumors (e.g., breast, prostate or ovarian cancer). Abnormal cellular proliferation conditions or cancers that may be treated in either adults or children include solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

In some preferred embodiments of the present invention, cancer includes colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, and Wilm's tumor.

In still further preferred embodiments of the present invention, cancer includes mullerian mixed tumor of the endometrium, infiltrating carcinoma of mixed ductal and lobular type, Wilm's tumor, mullerian mixed tumor of the ovary, serous cystadenocarcinoma, ovary adenocarcinoma (papillary serous type), ovary adenocarcinoma (endometrioid Type), metastatic infiltrating lobular carcinoma of breast, testis seminoma, prostate benign nodular hyperplasia, lung squamous cell carcinoma, lung large cell carcinoma, lung adenocarcinoma, endometrium adenocarcinoma (endometrioid type), infiltrating ductal carcinoma, skin basal cell carcinoma, breast infiltrating lobular carcinoma, fibrocystic disease, fibroadenoma, gleoma, chronic myeloid leukemia, liver hepatocellular carcinoma, mucinous carcinoma, schwannoma, kidney transitional cell carcinoma, Hashimoto's thyroiditis, metastatic infiltrating ductal carcinoma of breast, esophagus adenocarcinoma, thymoma, phyllodes tumor, rectum adenocarcinoma, osteosarcoma, colon adenocarcinoma, thyroid gland papillary carcinoma, leiomyoma, and stomach adenocarcinoma.

Examples of Inflammation

Examples of inflammation include, but are not limited to, systemic inflammatory conditions and conditions associated locally with migration and attraction of monocytes, leukocytes and/or neutrophils. Inflammation may result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subject's own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

In some preferred embodiments, the inflammation includes Non-Hodgkin's lymphoma, Wegener's granulomatosis, Hashimoto's thyroiditis, hepatocellular carcinoma, thymus atrophy, chronic pancreatitis, rheumatoid arthritis, reactive lymphoid hyperplasia, osteoarthritis, ulcerative colitis, papillary carcinoma, Crohn's disease, ulcerative colitis, acute cholecystitis, chronic cholecystitis, cirrhosis, chronic sialadenitis, peritonitis, acute pancreatitis, chronic pancreatitis, chronic Gastritis, adenomyosis, endometriosis, acute cervicitis, chronic cervicitis, lymphoid hyperplasia, multiple sclerosis, hypertrophy secondary to idiopathic thrombocytopenic purpura, primary IgA nephropathy, systemic lupus erythematosus, psoriasis, pulmonary emphysema, chronic pyelonephritis, and chronic cystitis.

Examples of Endocrine and Neuroendocrine Disorders

Examples of endocrine disorders include disorders of adrenal, breast, gonads, pancreas, parathyroid, pituitary, thyroid, dwarfism etc. The adrenal disorders include, but are not limited to, Addison's disease, hirutism, cancer, multiple endocrine neoplasia, congenital adrenal hyperplasia, and pheochromocytoma. The breast disorders include, but are not limited to, breast cancer, fibrocystic breast disease, and gynecomastia. The gonad disorders include, but are not limited to, congenital adrenal hyperplasia, polycystic ovarian syndrome, and turner syndrome. The pancreas disorders include, but are not limited to, diabetes (type I and type II), hypoglycemia, and insulin resistance. The parathyroid disorders include, but are not limited to, hyperparathyroidism, and hypoparathyroidism. The pituitary disorders include, but are not limited to, acromegaly, Cushing's syndrome, diabetes insipidus, empty sella syndrome, hypopituitarism, and prolactinoma. The thyroid disorders include, but are not limited to, cancer, goiter, hyperthyroid, hypothyroid, nodules, thyroiditis, and Wilson's syndrome. The examples of neuroendocrine disorders include, but are not limited to, depression and anxiety disorders related to a hormonal imbalance, catamenial epilepsy, menopause, menstrual migraine, reproductive endocrine disorders, gastrointestinal disorders such as, gut endocrine tumors including carcinoid, gastrinoma, and somatostatinoma, achalasia, and Hirschsprung's disease. In some embodiments, the endocrine and neuroendocrine disorders include nodular hyperplasia, Hashimoto's thyroiditis, islet cell tumor, and papillary carcinoma.

The endocrine and neuroendocrine disorders in children include endocrinologic conditions of growth disorder and diabetes insipidus. Growth delay may be observed with congenital ectopic location or aplasia/hypoplasia of the pituitary gland, as in holoprosencephaly, septo-optic dysplasia and basal encephalocele. Acquired conditions, such as craniopharyngiorna, optic/hypothalamic glioma may be present with clinical short stature and diencephalic syndrome. Precocious puberty and growth excess may be seen in the following conditions: arachnoid cyst, hydrocephalus, hypothalamic hamartoma and germinoma. Hypersecretion of growth hormone and adrenocorticotropic hormone by a pituitary adenoma may result in pathologically tall stature and truncal obesity in children. Diabetes insipidus may occur secondary to infiltrative processes such as langerhans cell of histiocytosis, tuberculosis, germinoma, post traumatic/surgical injury of the pituitary stalk and hypoxic ischemic encephalopathy.

Examples of Nutritional and Metabolic Disorders

The examples of nutritional and metabolic disorders include, but are not limited to, aspartylglusomarinuria, biotinidase deficiency, carbohydrate deficient glycoprotein syndrome (CDGS), Crigler-Najjar syndrome, cystinosis, diabetes insipidus, fabry, fatty acid metabolism disorders, galactosemia, gaucher, glucose-6-phosphate dehydrogenase (G6PD), glutaric aciduria, hurler, hurler-scheie, hunter, hypophosphatemia, 1-cell, krabbe, lactic acidosis, long chain 3 hydroxyacyl CoA dehydrogenase deficiency (LCHAD), lysosomal storage diseases, mannosidosis, maple syrup urine, maroteaux-lamy, metachromatic leukodystrophy, mitochondrial, morquio, mucopolysaccharidosis, neuro-metabolic, niemann-pick, organic acidemias, purine, phenylketonuria (PKU), pompe, pseudo-hurler, pyruvate dehydrogenase deficiency, sandhoff, sanfilippo, scheie, sly, tay-sachs, trimethylaminuria (fish-malodor syndrome), urea cycle conditions, vitamin D deficiency rickets, metabolic disease of muscle, inherited metabolic disorders, acid-base imbalance, acidosis, alkalosis, alkaptonuria, alpha-mannosidosis, amyloidosis, anemia, iron-deficiency, ascorbic acid deficiency, avitaminosis, beriberi, biotinidase deficiency, deficient glycoprotein syndrome, carnitine disorders, cystinosis, cystinuria, fabry disease, fatty acid oxidation disorders, fucosidosis, galactosemias, gaucher disease, gilbert disease, glucosephosphate dehydrogenase deficiency, glutaric academia, glycogen storage disease, hartnup disease, hemochromatosis, hemosiderosis, hepatolenticular degeneration, histidinemia, homocystinuria, hyperbilirubinemia, hypercalcemia, hyperinsulinism, hyperkalemia, hyperlipidemia, hyperoxaluria, hypervitaminosis A, hypocalcemia, hypoglycemia, hypokalemia, hyponatremia, hypophosphotasia, insulin resistance, iodine deficiency, iron overload, jaundice, chronic idiopathic, leigh disease, Lesch-Nyhan syndrome, leucine metabolism disorders, lysosomal storage diseases, magnesium deficiency, maple syrup urine disease, MELAS syndrome, menkes kinky hair syndrome, metabolic syndrome X, mucolipidosis, mucopolysacchabridosis, Niemann-Pick disease, obesity, ornithine carbamoyltransferase deficiency disease, osteomalacia, pellagra, peroxisomal disorders, porphyria, erythropoietic, porphyries, progeria, pseudo-gaucher disease, refsum disease, reye syndrome, rickets, sandhoff disease, tangier disease, Tay-sachs disease, tetrahydrobiopterin deficiency, trimethylaminuria (fish odor syndrome), tyrosinemias, urea cycle disorders, water-electrolyte imbalance, wernicke encephalopathy, vitamin A deficiency, vitamin B12 deficiency, vitamin B deficiency, wolman disease, and zellweger syndrome.

In some preferred embodiments, the metabolic diseases include diabetes and obesity.

Examples of Hematolymphoid System

A hematolymphoid system includes hemic and lymphatic diseases. A “hematological disorder” includes a disease, disorder, or condition which affects a hematopoietic cell or tissue. Hematological disorders include diseases, disorders, or conditions associated with aberrant hematological content or function. Examples of hematological disorders include disorders resulting from bone marrow irradiation or chemotherapy treatments for cancer, disorders such as pernicious anemia, hemorrhagic anemia, hemolytic anemia, aplastic anemia, sickle cell anemia, sideroblastic anemia, anemia associated with chronic infections such as malaria, trypanosomiasis, HIV, hepatitis virus or other viruses, myelophthisic anemias caused by marrow deficiencies, renal failure resulting from anemia, anemia, polycethemia, infectious mononucleosis (IM), acute non-lymphocytic leukemia (ANLL), acute Myeloid Leukemia (AML), acute promyelocytic leukemia (APL), acute myelomonocytic leukemia (AMMol), polycethemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalessemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as plasmodium, chemical injuries from, e.g., lead poisoning, and hypersplenism.

Lymphatic diseases include, but are not limited to, lymphadenitis, lymphagiectasis, lymphangitis, lymphedema, lymphocele, lymphoproliferative disorders, mucocutaneous lymph node syndrome, reticuloendotheliosis, splenic diseases, thymus hyperplasia, thymus neoplasms, tuberculosis, lymph node, pseudolymphoma, and lymphatic abnormalities.

In some preferred embodiments, the disorders of hematolymphoid system include, non-Hodgkin's lymphoma, chronic lymphocytic leukemia, and reactive lymphoid hyperplasia.

Examples of CNS Diseases

The examples of CNS diseases include, but are not limited to, neurodegenerative diseases, drug abuse such as, cocaine abuse, multiple sclerosis, schizophrenia, acute disseminated encephalomyelitis, transverse myelitis, demyelinating genetic diseases, spinal cord injury, virus-induced demyelination, progressive multifocal leucoencephalopathy, human lymphotrophic T-cell virus I (HTLVI)-associated myelopathy, and nutritional metabolic disorders.

In some preferred embodiments, the CNS diseases include Parkinson disease, Alzheimer's disease, cocaine abuse, and schizophrenia.

Examples of Neurodegenerative Diseases

Neurodegenerative diseases in the methods of the present invention include, but are not limited to, Alzheimer's disease, Pick's disease, diffuse lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-dementia complex of guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, kuru and fatal familial insomnia), Alexander disease, alper's disease, amyotrophic lateral sclerosis, ataxia telangiectasia, batten disease, canavan disease, cockayne syndrome, corticobasal degeneration, Creutzfeldt-Jakob disease, Huntington disease, Kennedy's disease, Krabbe disease, lewy body dementia, Machado-Joseph disease, spinocerebellar ataxia type 3, multiple sclerosis, multiple system atrophy, Parkinson disease, Pelizaeus-Merzbacher Disease, Refsum's disease, Schilder's disease, Spielmeyer-Vogt-Sjogren-Batten disease, Steele-Richardson-Olszewski disease, and tabes dorsalis.

Examples of Disorders of Urinary Tract

Disorders of urinary tract in the methods of the present invention include, but are not limited to, disorders of kidney, ureters, bladder, and urethera. For example, urethritis, cystitis, pyelonephritis, renal agenesis, hydronephrosis, polycystic kidney disease, multicystic kidneys, low urinary tract obstruction, bladder exstrophy and epispadias, hypospadias, bacteriuria, prostatitis, intrarenal and peripheral abscess, benign prostate hypertrophy, renal cell carcinoma, transitional cell carcinoma, Wilm's tumor, uremia, and glomerolonephritis.

Examples of Respiratory Diseases

The respiratory diseases and conditions include, but are not limited to, asthma, chronic obstructive pulmonary disease (COPD), adenocarcinoma, adenosquamous carcinoma, squamous cell carcinoma, large cell carcinoma, cystic fibrosis (CF), dispnea, emphysema, wheezing, pulmonary hypertension, pulmonary fibrosis, hyper-responsive airways, increased adenosine or adenosine receptor levels, pulmonary bronchoconstriction, lung inflammation and allergies, and surfactant depletion, chronic bronchitis, bronchoconstriction, difficult breathing, impeded and obstructed lung airways, adenosine test for cardiac function, pulmonary vasoconstriction, impeded respiration, acute respiratory distress syndrome (ARDS), administration of certain drugs, such as adenosine and adenosine level increasing drugs, and other drugs for, e.g. treating supraventricular tachycardia (SVT), and the administration of adenosine stress tests, infantile respiratory distress syndrome (infantile RDS), pain, allergic rhinitis, decreased lung surfactant, decreased ubiquinone levels, or chronic bronchitis, among others.

Examples of Disorders of Female Genital System

The disorders of the female genital system include diseases of the vulva, vagina, cervix uteri, corpus uteri, fallopian tube, and ovary. Some of the examples include, adnexal diseases such as, fallopian tube disease, ovarian disease, leiomyoma, mucinous cystadenocarcinoma, serous cystadenocarcinoma, parovarian cyst, and pelvic inflammatory disease; endometriosis; genital neoplasms such as, fallopian tube neoplasms, uterine neoplasms, vaginal neoplasms, vulvar neoplasms, and ovarian neoplasms; gynatresia; genital herpes; infertility; sexual dysfunction such as, dyspareunia, and impotence; tuberculosis; uterine diseases such as, cervix disease, endometrial hyperplasia, endometritis, hematometra, uterine hemorrhage, uterine neoplasms, uterine prolapse, uterine rupture, and uterine inversion; vaginal diseases such as, dyspareunia, hematocolpos, vaginal fistula, vaginal neoplasms, vaginitis, vaginal discharge, and candidiasis or vulvovaginal; vulvar diseases such as, kraurosis vulvae, pruritus, vulvar neoplasm, vulvitis, and candidiasis; and urogenital diseases such as urogenital abnormalities and urogenital neoplasms.

Examples of Disorders of Male Genital System

The disorders of the male genital system include, but are not limited to, epididymitis; genital neoplasms such as, penile neoplasms, prostatic neoplasms, and testicular neoplasms; hematocele; genital herpes; hydrocele; infertility; penile diseases such as, balanitis, hypospadias, peyronie disease, penile neoplasms, phimosis, and priapism; prostatic diseases such as, prostatic hyperplasia, prostatic neoplasms, and prostatitis; organic sexual dysfunction such as, dyspareunia, and impotence; spermatic cord torsion; spermatocele; testicular diseases such as, cryptorchidism, orchitis, and testicular neoplasms; tuberculosis; varicocele; urogenital diseases such as, urogenital abnormalities, and urogenital neoplasms; and fournier gangrene.

Examples of Cardiovascular Disorders (CVS)

The cardiovascular disorders include those disorders that can either cause ischemia or are caused by reperfusion of the heart. Examples include, but are not limited to, atherosclerosis, coronary artery disease, granulomatous myocarditis, chronic myocarditis (non-granulomatous), primary hypertrophic cardiomyopathy, peripheral artery disease (PAD), stroke, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, but not limited to, tissue damage related to PARP activation. In some preferred embodiments of the present invention, CVS diseases include, atherosclerosis, granulomatous myocarditis, myocardial infarction, myocardial fibrosis secondary to valvular heart disease, myocardial fibrosis without infarction, primary hypertrophic cardiomyopathy, and chronic myocarditis (non-granulomatous).

Methods of Treatment

The methods provided by the invention may comprise the administration of the compounds of formula I, II and/or their preferred embodiments. The compounds can also be administered in combination with other therapies. The choice of therapy that can be co-administered with the compositions of the invention will depend, in part, on the condition being treated. For example, for treating acute myeloid leukemia, compound of some embodiments of the invention can be used in combination with radiation therapy, monoclonal antibody therapy, chemotherapy, bone marrow transplantation, or a combination thereof.

An effective therapeutic amount of the PARP inhibitors is administered to a patient, preferably a mammal and more preferably a human, to affect a pharmacological activity involving inhibition of a PARP enzyme. As such, PARP inhibitors of the present invention may be useful in treating or preventing a variety of diseases and illnesses including neural tissue damage resulting from cell damage or death due to necrosis or apoptosis, cerebral ischemia and reperfusion injury or neurodegenerative diseases in an animal. In addition, compounds of the present invention can also be used to treat a cardiovascular disorder in an animal, by administering an effective amount of the PARP inhibitor to the animal. Further still, the compounds of the invention can be used to treat cancer and to radiosensitize or chemosensitize tumor cells.

In some embodiments of the present invention, the PARP inhibitors can be used to stimulate damaged neurons, promote neuronal regeneration, prevent neurodegeneration and/or treat a neurological disorder. The PARP inhibitors inhibit PARP activity and, thus, are useful for treating neural tissue damage, particularly damage resulting from cancer, cardiovascular disease, cerebral ischemia and reperfusion injury or neurodegenerative diseases in animals. The PARP inhibitors in the present invention can be useful for treating cardiac tissue damage, particularly damage resulting from cardiac ischemia or caused by reperfsion injury in a patient. The compounds of the invention can be particularly useful for treating cardiovascular disorders selected from the group consisting of: coronary artery disease, such as atherosclerosis; angina pectoris; myocardial infarction; myocardial ischemia and cardiac arrest; cardiac bypass; and cardiogenic shock.

In another aspect, the PARP inhibitors in the present invention can be used to treat cancer, and to radiosensitize and/or chemosensitize tumor cells. The PARP inhibitors of the present invention can be “anti-cancer agents,” which term also encompasses “anti-tumor cell growth agents” and “anti-neoplastic agents.” For example, the PARP inhibitors of the invention are useful for treating cancers, and radiosensitizing and/or chemosensitizing tumor cells in cancers.

Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: Hematoporphyrin derivatives, Photofrin, benzoporphyrin derivatives, NPe6, tin etioporphyrin SnET2, pheoborbide-α, bacteriochlorophyll-α, naphthalocyanines, phthalocyanines, zinc phthalocyanine, and therapeutically effective analogs and derivatives of the same.

Radiosensitizers can be administered in conjunction with a therapeutically effective amount of one or more other PARP inhibitors, including but not limited to: PARP inhibitors which promote the incorporation of radiosensitizers to the target cells; PARP inhibitors which control the flow of therapeutics, to nutrients, and/or oxygen to the target calls. Similarly, chemosensitizers are also known to increase the sensitivity of cancerous cells to the toxic effects of chemotherapeutic compounds. Exemplary chemotherapeutic agents that can be used in conjunction with PARP inhibitors include, but are not limited to, adriamycin, camptothecin, dacarbazine, carboplatin, cisplatin, daunorubicin, docetaxel, doxorubicin, interferon (alpha, beta, gamma), interleukin 2, innotecan, paclitaxel, streptozotocin, temozolomide, topotecan, and therapeutically effective analogs and derivatives of the same. In addition, other therapeutic agents which can be used in conjunction with a PARP inhibitors include, but are not limited to, 5-fluorouracil, leucovorin, 5′-amino-5′-deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO.

Formulations, Routes of Administration, and Effective Doses

Another aspect of the present invention relates to formulations and routes of administration for pharmaceutical compositions comprising compound of formula I, its preferred embodiments, II and/or IIa. Such pharmaceutical compositions can be used to treat cancer in the methods described in detail above.

The compound of formula I, its preferred embodiments, II and/or IIa may be provided as a prodrug and/or may be allowed to interconvert to its form in vivo after administration. That is, either the compounds or their pharmaceutically acceptable salts may be used in developing a formulation for use in the present invention. Further, in some embodiments, the compound may be used in combination with one or more other compounds or in one or more other forms. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

In compositions comprising combinations of a compound of formula I, its preferred embodiments, II and/or IIa and another active agent may be effective. The two compounds and/or forms of a compound may be formulated together, in the same dosage unit e.g. in one cream, suppository, tablet, capsule, or packet of powder to be dissolved in a beverage; or each form may be formulated in separate units, e.g, two creams, suppositories, tablets, two capsules, a tablet and a liquid for dissolving the tablet, a packet of powder and a liquid for dissolving the powder, etc.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions may contain compound of formula I, its preferred embodiments, II and/or IIa with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol, 23: 238-252 (1965) and Szoka et al., Proc. Natl. Acad. Sci 75: 4194-4198 (1978), incorporated herein by reference. Ligands may also be attached to the liposomes to direct these compositions to particular sites of action. Compounds of this invention may also be integrated into foodstuffs, e.g, cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

For injection, the inhibitors of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P. These compounds may also be formulated for transmucosal administration, buccal administration, for administration by inhalation, for parental administration, for transdermal administration, and rectal administration.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions suitable for use in the present invention include compositions wherein the active ingredients are present in an effective amount, i.e., in an amount effective to achieve therapeutic and/or prophylactic benefit in at least one of the cancers described herein. The actual amount effective for a particular application will depend on the condition or conditions being treated, the condition of the subject, the formulation, and the route of administration, as well as other factors known to those of skill in the art. Determination of an effective amount of a compound of formula I, its preferred embodiments, II and/or IIa is well within the capabilities of those skilled in the art, in light of the disclosure herein, and will be determined using routine optimization techniques.

The following preparations and examples serve to illustrate the invention. The examples as described below are in no way intended to limit or narrow the scope of the instant invention. Further, it can be appreciated to one of ordinary skill in the art that many changes and modifications can be made thereto without departing from the spirit or scope of the appended claims, and such changes and modifications are contemplated within the scope of the instant invention.

### EXAMPLES

PARP-1 is purified from calf thymus as reported earlier (Molinet et al. (1993) EMBO J. 12:2109-2117). Alternatively recombinant PARP-1 is isolated from Sodoptera Fugiperda (Sf9) cells infected with recombinant baculovirus, expressing the human PARP-1 gene, constructed according to the instructions of Pharmingen. The cDNA of the amino acid exchange mutant R34G and R138 il of PARP-1 is created by the mega primer method (Kannann et al. (1989) Nucl Acids Res 17:5404). The mutated gene is cloned into the transfer vector pV 1392 and the recombinant virus is generated by the Baculogold technology of Pharmigen. The mutated proteins are expressed in Sf9 cells, purified and assayed as reported (Huang et al. (2004) Biochemistry 43:217-223; Kirsten et al. (2004) Methods in Molecular Biology 287, Epigenetics Protocols 137-149).

Poly(ADP-ribose)glycohydrolase (PARG) is purchased from Biomol or Alexis Co, which enzymes are equal in performance. Jurkat cells are cultured as reported (Buday et al. (1996) J Biol Chem 271:6159-6163) and nuclei are prepared by a published method (Smirnova et al. (2000) J Biol Chem 275:9377-9384). Enzymatic assays for PARP-1 are performed as published (deMurcia (2000) From DNA-damage and Stress Signalling to Cell Death: Poly ADP-ribosylation Reactions). PARG activity is analyzed with polyADP-ribosylated PARP-1 as substrate, containing long chains synthesized with spermine as cofactor (Kun et al. (2004) Biochemistry 43:210-216), or short chains where the cofactor is histone H1. The polyADP-ribose is labeled either with 3H or by biotinylated-NAD. PARG activity is quantitatively measured either by assaying the liberated 3H-ADP-ribose by TLC (Kirsten et al. (1991) Exp Cell Res 194:1-8) (chromatography carried out on PEI-cellulose sheets with 0.9 M acetic acid and 0.3 M LiCl as solvent) or by immunoassay of the remaining biotinylated (ADP-ribose)ₙ (Bakondi et al. (2004) Exp Dermatol 13:170-178). All other reagents are of highest analytical purity.

### Example 1

### Enzymatic Activities of Wild Type Arginine-34 and Arginine-138 Mutant PARP-1

Assays are carried out as described (Kun et al. (2004) Biochemistry, 43:210-216) in triplicate (200 μM 3H labeled NAD+, 28 dpm/pmol, 0.5 pmol PARP-1, 3 mM spermine, pH 7.3 t=7.5 min). Identical results are obtained when ATP is replaced by its non-hydrolysable analog (Kun et al. (2004) Biochemistry, 43:210-216).

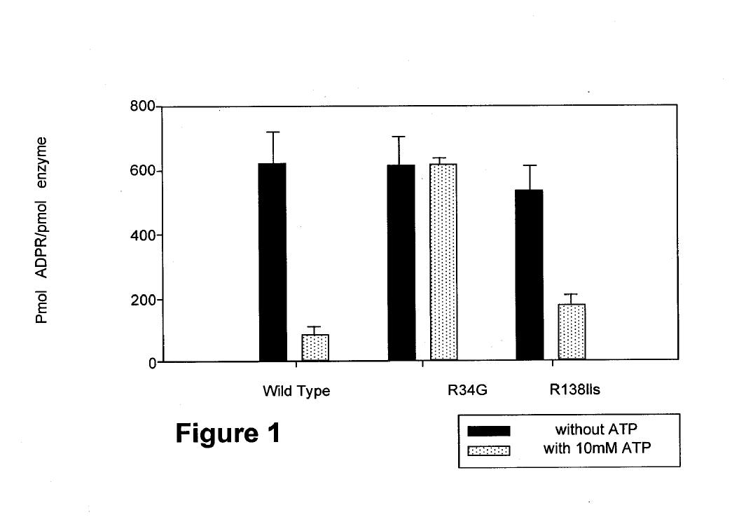

The effect of replacing arginine-34 by glycine in Zn²⁺ finger 1 of PARP-1 is shown in FIG. 1. While the total enzymatic activity of PARP-1 is not affected by this mutation, the inhibitory action of ATP (or its non-hydrolysable analog) is abolished. These results show that only PARP-1 is sensitive to regulation by ATP. Mutation of arginine-138 to isoleucine in Zn²⁺ finger 2 has negligible effect on the inhibitory action of ATP, confirming our observation that arginine-34 of Zn²⁺ finger 1 is the site of ATP interaction with PARP-1.

### Example 2

### Effect of ATP on the PARP-1 Activity of Jurkat Cell Nuclei

Nuclei equivalent to 2×105 Jurkat cells are pre incubated in the presence of various concentrations of ATP. Then PARP activities are assayed by admixing biotinylated-NAD (5 μM final conc.) and incubating for ten minutes. After separating the proteins on a 8% SDS-PAGE gel, the nitrocellulose-blotted, labeled proteins are detected by incubating with streptavidine-HPO complex (1 μg/ml) and by fluorography. Triplicate results are expressed as densitometric units.

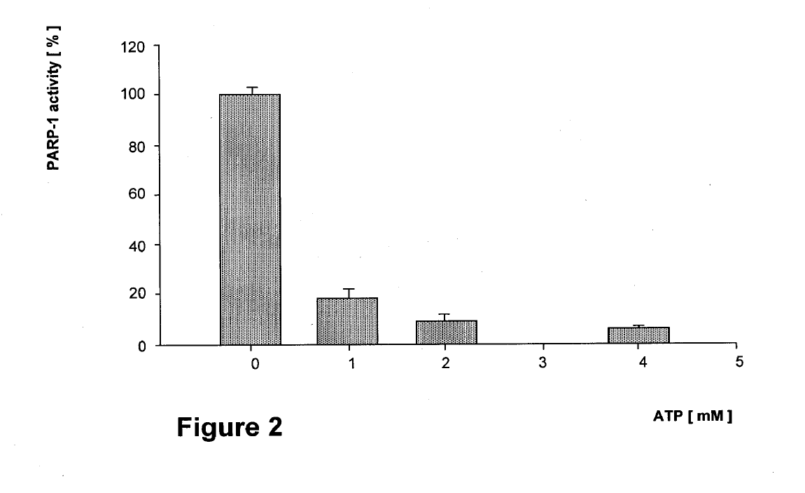

The action of externally added ATP (or its non-hydrolysable analog) on PARP-1 activity of isolated Jurkat cell nuclei is shown in FIG. 2. A precipitous inhibition of PARP-1 activity is apparent which may be even larger in nuclei than reported for the isolated enzyme since Ki of ATP for the pure enzyme is between 2 to 2.5 mM (3), but in nuclei 1 mM of ATP already inhibits PARP-1 by 80%. This difference may be due either to the higher sensitivity of structurally associated PARP-1 in nuclei or to some loss of diffusible ds DNA-s (Kun et al. (2002) J Biol Chem 277:39066-39069; Kun et al. (2004) Biochemistry 43:210-216) that could occur during isolation of nuclei.

### Example 3

### Effect of BCNU on the ATP Sensitivity of PARP-1 Activity of Jurkat Cell Nuclei

Experiments in triplicate are carried out as described in FIG. 2, with the exception that pre incubation is done with 400 nM of BCNU for 30 minutes. First bar shows the PARP activity of BCNU non-treated nuclei.

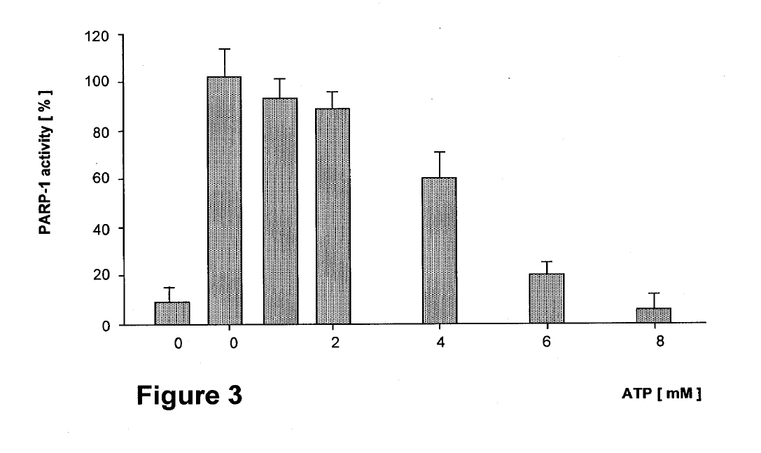

The consequences of DNA damage by BCNU on PARP-1 activation and the suppression of this pathophysiologically significant process by ATP is illustrated in FIG. 3. The response to DNA damage by BCNU as assayed by PARP-1 activity is completely removed by externally added ATP, demonstrating that the action of BCNU is dependent on the bioenergetic competence of the target cancer cell.

### Example 4

### Effect of ATP on the Glycohydrolase Activity of Jurkat Cell Nuclear Extract

Poly(ADP-ribosylated)-PARP-1 (2 μg protein is incubated with 50 μM of biotinylated-NAD as described above) and is attached to the walls of 96-well plates. (Kannann et al. (1989) Nucl. Acids Res. 17:5404). Jurkat cell nuclear extract (50 μg protein) is incubated either in the presence (Δ-Δ) or absence (●-●) of 10 mM of ATP for various times indicated on the abscissa. The amount of PAR remaining attached to the wells is assayed with the Trevigen assay (ordinate) in triplicate.

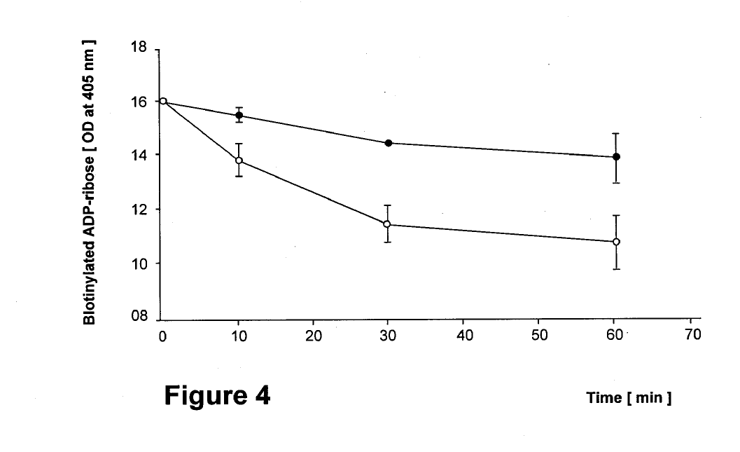

When Jurkat cell extracts are incubated with polyADP-ribosylated PARP-1, containing long polymers (50 ADP-ribose units), the decay of the polymer is apparent, which is the consequence of PARG activity present in cell extracts (FIG. 4). Addition of 8 mM of ATP significantly accelerates PARG activity.

### Example 5

### Effect of the Chain-Length of the PAR Polymer on the ATP Sensitivity of Purified PARG

Short (Δ-Δ)-or long (●-●)-chain PAR-PARP molecules are prepared as described in the methods and attached to the surface of assay wells. Purified PARG (15 mU/assay) is added to the wells in the presence of various concentrations of ATP and incubated for 45 minutes. The attached amount of polymers is determined in triplicate experiments (zero minute values for short chains were 0.5 OD, and 1.8 OD for long chain polymers).

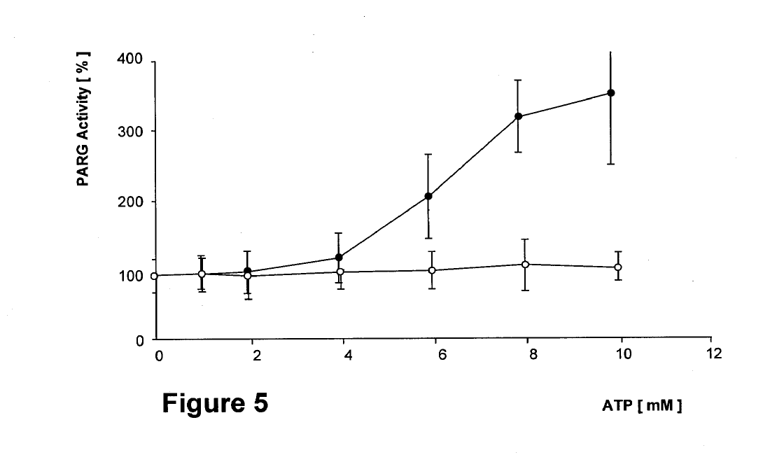

The discrimination between long and short ADP-ribose oligomers with respect to susceptibility to PARG is shown in FIG. 5, from which it is apparent that the degradation of short oligomers is not accelerated by ATP, only the decay of longer oligomers (average chain length 50 ADPR) is accelerated.

### Example 6

### The Effect of ATP on PARG Activity as a Function of Substrate (PAR) Concentration

PARG (15 mU/assay) is incubated with various concentrations of 32P-PAR (long chain polymer) in the presence or absence of 6 mM of ATP for 45 minutes and the amount of liberated 32P-ADP-ribose is determined by TLC and liquid scintillation of the cut out portions of the plate located by autoradiography. Experiments are performed in triplicate.

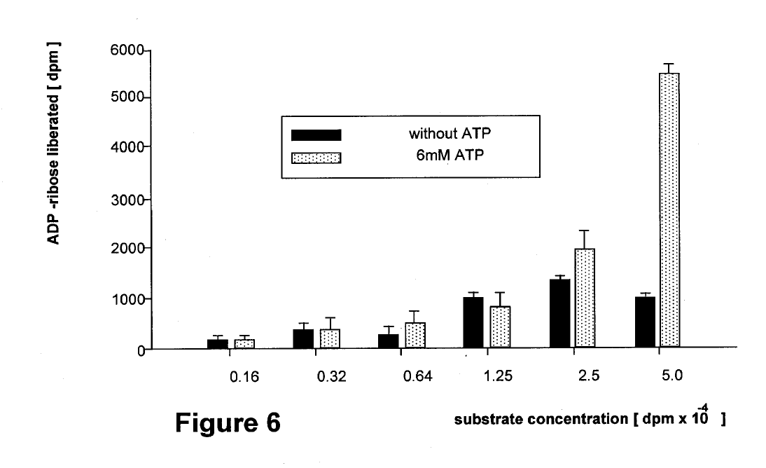

The PARG catalyzed reaction is also followed by measuring the liberation of 3H-ADP-ribose as shown in FIG. 6, demonstrating the activation of the exonucleotidase activity of PARG when long chain polymers are the substrate.

### Example 7

### Use of a Compound in the Treatment of Solid Tumor Colon Cancer

A subject suffering from solid tumor colon cancer is treated with a therapeutically effective amount of compound of formula 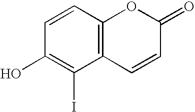
where the compound is administered orally or parenterally. After few days, the symptoms of the cancer are markedly reduced.

### Example 8

### Use of a Compound in the Treatment of Solid Tumor Colon Cancer

The method of example 7 is repeated, except that the patient suffering from the cancer is administered with a compound of formula

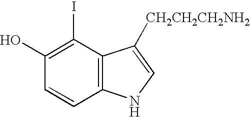

A similar result is obtained.

### Example 9

### Use of a Compound in the Treatment of Solid Tumor Colon Cancer

The method of example 7 is repeated, except that the patient suffering from the cancer is administered with a compound of formula

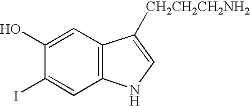

A similar result is obtained.

### Example 10

### Use of a Compound in the Treatment of Solid Tumor Colon Cancer

The method of example 7 is repeated, except that the patient suffering from the cancer is administered with a compound of formula

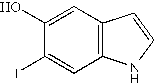

A similar result is obtained.

### Example 11

### Use of a Compound in the Treatment of Solid Tumor Colon Cancer

The method of example 7 is repeated, except that the patient suffering from the cancer is administered with a compound of formula

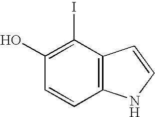

A similar result is obtained.

### Example 12

### Use of a Compound in the Treatment of Solid Tumor Colon Cancer

The method of example 7 is repeated, except that the patient suffering from the cancer is administered with a compound of formula

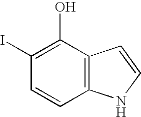

A similar result is obtained.

### Example 13

### Use of a Compound in the Treatment of Inflammation

A subject suffering from inflammation is treated with a therapeutically effective amount of compound of formula 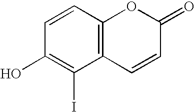
where the compound is administered orally or parenterally. After few days, the symptoms of inflammation are markedly reduced.

### Example 14

### Use of a Compound in the Treatment of Inflammation

The method of example 13 is repeated, except that the patient suffering from inflammation is administered with a compound of formula

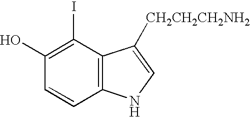

A similar result is obtained.

### Example 15

### Use of a Compound in the Treatment of Inflammation

The method of example 13 is repeated, except that the patient suffering from inflammation is administered with a compound of formula

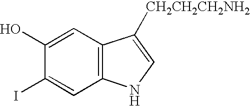

A similar result is obtained.

### Example 16

### Use of a Compound in the Treatment of Inflammation

The method of example 13 is repeated, except that the patient suffering from inflammation is administered with a compound of formula

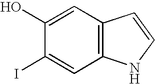

A similar result is obtained.

### Example 17

### Use of a Compound in the Treatment of Inflammation

The method of example 13 is repeated, except that the patient suffering from inflammation is administered with a compound of formula

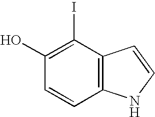

A similar result is obtained.

### Example 18

### Use of a Compound in the Treatment of Inflammation

The method of example 13 is repeated, except that the patient suffering from inflammation is administered with a compound of formula

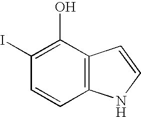

A similar result is obtained.

### Example 19

### Use of a Compound in the Treatment of CNS Disease

A subject suffering from CNS disease is treated with a therapeutically effective amount of compound of formula 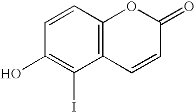
where the compound is administered orally or parenterally. After few days, the symptoms of CNS disease are markedly reduced.

### Example 20

### Use of a Compound in the Treatment of CNS Disease

The method of example 19 is repeated, except that the patient suffering from CNS disease is administered with a compound of formula

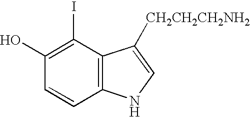

A similar result is obtained.

### Example 21

### Use of a Compound in the Treatment of CNS Disease

The method of example 19 is repeated, except that the patient suffering from CNS disease is administered with a compound of formula

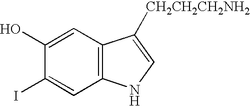

A similar result is obtained.

### Example 22

### Use of a Compound in the Treatment of CNS Disease

The method of example 19 is repeated, except that the patient suffering from CNS disease is administered with a compound of formula

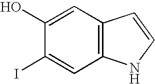

A similar result is obtained.

### Example 23

### Use of a Compound in the Treatment of CNS Disease

The method of example 19 is repeated, except that the patient suffering from CNS disease is administered with a compound of formula

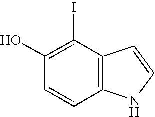

A similar result is obtained.

### Example 24

### Use of a Compound in the Treatment of CNS Disease

The method of example 19 is repeated, except that the patient suffering from CNS disease is administered with a compound of formula

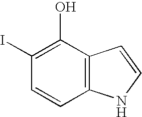

A similar result is obtained.

What is claimed is:

1. A method of modulating PARP-1 activity in a mammal comprising administering to a mammal an effective amount of an organic aromatic compound having from 4 to about 35 carbon atoms, wherein said organic aromatic compound is capable of binding the arginine-34 moiety located in Zinc finger-1 of the PARP-1 enzyme and wherein said organic aromatic compound has electron donating capabilities such that it's iπ-electron system will interact with the positively charged (cationic) guanidinium moiety of the specific arginine-34 residue of the Zinc-1 finger of PARP-1 where when said aromatic compound contains a heterocyclic ring containing a nitrogen atom/said ring does not contain a carbonyl moiety and does not contain a lactam structure and the substituents do not contain a benzamide or lactam structure.

2. The method as recited in claim 1 wherein an organic aromatic compound is selected from the group consisting of formula I and II 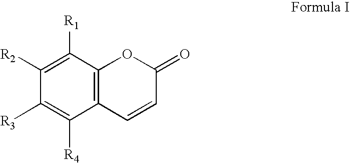
wherein R₁, R₂, R₃ and R₄ are independently selected from the group consisting of H, halogen, optionally substituted hydroxy, substituted amine, optionally substituted lower alkyl, optionally substituted phenyl, optionally substituted C₄-C₁₀ heteroaryl and optionally substituted C₃-C₈cycloalkyl or a salt, solvate, isomer, tautomers, metabolite, or prodrug thereof. 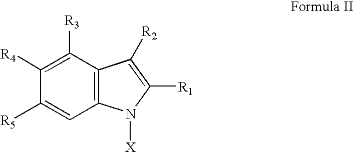
wherein R₁, R₂, R₃, R₄ and R₅ are independently selected from the group consisting of H, halogen, nitro, nitroso, optionally substituted hydroxy, optionally substituted lower alkyl, optionally substituted amine, optionally substituted phenyl, optionally substituted C₄-C₁₀ heteroaryl and optionally substituted C₃-C₈ cycloalkyl; X is H, N-oxide or optionally substituted alkyl or a salt, solvate, isomer, tautomers, metabolite, or prodrug thereof.

3. The method as recited in claim 1 wherein said modulating is inhibiting.

4. The method as recited in claim 1 wherein said inhibiting is irreversible.

5. A compound of formula 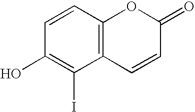
or a salt, solvate, isomer, tautomers, metabolite, or prodrug thereof.

6. A compound of formula IIa 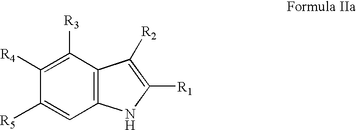
wherein R₁, R₂, R₃, R₄ and R₅ are independently selected from the group consisting of iodo, hydroxyl, nitro, nitroso, and optionally substituted amine or a salt, solvate, isomer, tautomers, metabolite, or prodrug thereof.

7. The compound as recited in claim 6 wherein R₁, R₂ and R₅ are hydrogen, R₃ is hydroxyl and R₄ is iodo.

8. The compound as recited in claim 6 wherein R₁, R₂ and R₅ are hydrogen, R₄ is hydroxyl and R₃ is iodo.

9. The compound as recited in claim 6 wherein R₁, R₂ and R₃ is hydrogen, R₅ is iodo and R₄ is hydroxyl.

10. The compound as recited in claim 6 wherein R₂ is aminopropyl, R₃ is iodo and R₄ is hydroxyl and R₅ is hydrogen.

11. The compound as recited in claim 6 wherein R₂ is aminopropyl, R₃ is hydrogen and R₄ is hydroxyl and R₅ is iodo.

12. A pharmaceutical composition comprising an effective amount of at least one compound as recited in claim 5 or 6 with a pharmaceutically acceptable carrier, excipient and/or dilutent.

13. A method of treatment of a PARP mediated disease comprising administering to a subject in need thereof a therapeutically effective amount of an organic aromatic compound having from 4 to about 35 carbon atoms, wherein said organic aromatic compound is capable of binding the arginine-34 moiety located in Zinc finger-1 of the PARP-1 enzyme and wherein said organic aromatic compound has electron donating capabilities such that it's π-electron system will interact with the positively charged (cationic) guanidinium moiety of the specific arginine-34 residue of the Zinc-1 finger of PARP-1 where when said aromatic compound contains a heterocyclic ring containing a nitrogen atom, said ring does not contain a carbonyl moiety and does not contain a lactam structure and the substituents do not contain a benzamide or lactam structure.

14. The method as recited in claim 13 where an organic aromatic compound is selected from the group consisting of formula I and II 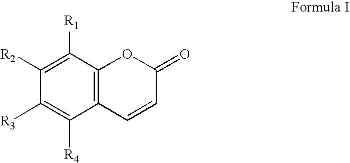
wherein R₁, R₂, R₃ and R₄ are independently selected from the group consisting of H, halogen, optionally substituted hydroxy, substituted amine, optionally substituted lower alkyl, optionally substituted phenyl, optionally substituted C₄-C₁₀ heteroaryl and optionally substituted C₃-C₈cycloalkyl or a salt, solvate, isomer, tautomers, metabolite, or prodrug thereof. 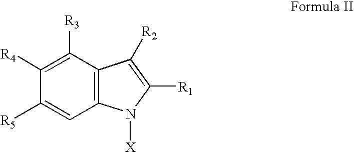
wherein R₁, R₂, R₃, R₄ and R₅ are independently selected from the group consisting of H, halogen, nitro, nitroso, optionally substituted hydroxy, optionally substituted lower alkyl, optionally substituted amine, optionally substituted phenyl, optionally substituted C₄-C₁₀ heteroaryl and optionally substituted C₃-C₈ cycloalkyl; X is H, N-oxide or optionally substituted alkyl or a salt, solvate, isomer, tautomers, metabolite, or prodrug thereof.
